(19) United States
(12) Patent Application Publication
(10) Pub. No.: US 2021/0386656 A1
(43) Pub. Date: Dec. 16, 2021

(54) LINKABLE POLYSILOXANES IN COSMETIC PRODUCTS

(71) Applicant: Coty Inc., New York, NY (US)

(72) Inventors: Yan Wang, Parsippany, NJ (US); Yuemei Zhang, Ramsey, NJ (US); Donald Rick, Dumont, NJ (US)

(21) Appl. No.: 17/286,347

(22) PCT Filed: Dec. 3, 2019
(86) PCT No.: PCT/US2019/064193

§ 371 Date: Apr. 16, 2021

Related U.S. Application Data
(60) Provisional application No. 62/784,130, filed on Dec. 21, 2018.

(51) Int. Cl. A61K 8/895; A61K 8/892; A61K 8/19

(52) U.S. Cl. CPC A61K 8/895; A61K 2800/95; A61K 8/19; A61K 8/892

(57) ABSTRACT

The present disclosure provides a linkable cosmetic composition. The composition includes a vinyl pendant siloxane component and a hydrogen pendant siloxane component. The composition further includes a catalyst and a solvent.

### CLAIM OF PRIORITY

This patent application claims the benefit of priority to U.S. Application Ser. No. 62/784,130, filed Dec. 21, 2018, which is incorporated by reference herein in its entirety.

### BACKGROUND

Methods for reducing the appearance of skin imperfections, for example wrinkles, fine lines, age spots, enlarged pores or scars, include invasive and non-invasive methods and formulations. Invasive techniques, such as surgery, fillers (e.g., Restylane, Juvederm), laser resurfacing or BOTOX, may provide longer-lasting effects and can treat prominent imperfections. However, many consumers either cannot afford or do not wish undergo such drastic cosmetic treatments. There is therefore a need to develop products which can help to reduce the appearance of skin imperfections using facile non-invasive methods.

### SUMMARY OF THE DISCLOSURE

The present disclosure provides a linkable cosmetic composition. The composition includes a vinyl pendant siloxane component and a hydrogen pendant siloxane component. The composition further includes a catalyst and a solvent.

The present disclosure further provides a substrate having a linkable cosmetic composition disposed thereon. The composition includes a vinyl pendant siloxane component and a hydrogen pendant siloxane component. The composition further includes a catalyst and a solvent.

The present disclosure further provides a cross-linked composition of a linkable composition. The linkable composition includes a vinyl pendant siloxane component and a hydrogen pendant siloxane component. The composition further includes a catalyst and a solvent.

The present disclosure further provides a method of using a linkable composition. The composition includes a vinyl pendant siloxane component and a hydrogen pendant siloxane component. The composition further includes a catalyst and a solvent. The method includes disposing a quantity of the composition on a substrate comprising keratin. The method further includes linking the linkable cosmetic composition.

Disclosed herein are linkable compositions that are capable of forming a linked polysiloxane network that is capable of reducing the appearance of wrinkles on a user's skin. In some embodiments, the linkable composition can be easily applied in two phases and can reduce or even eliminate the appearance of wrinkles on a user's skin. The linkable compositions can be a stand-alone composition such as an anti-wrinkle solution. The linkable composition can further be incorporated into a cosmetic product such as a foundation, a mascara, and a skincare product.

According to various embodiments, a the polysiloxane networks described herein can form a network having sufficient cohesive strength that can allow the network to be disposed and effective for a long time. Further, if the polysiloxane network is colored the network is strong enough to help to prevent smudging.

### DETAILED DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

The term “room temperature” as used herein refers to a temperature of about 15° C. to 40° C.

The polymers described herein can terminate in any suitable way. In some embodiments, the polymers can terminate with an end group that is independently chosen from a suitable polymerization initiator, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

According to various embodiments of the present disclosure, a linkable or curable cosmetic composition can include various components, combinations thereof, and sub-combinations thereof. Examples of suitable components can include a vinyl pendent polysiloxane component; a hydrogen pendent polysiloxane component; a catalyst component; and a solvent component.

The vinyl pendent polysiloxane component can be present in the linkable composition in any suitable weight percentage (wt %). For example, the vinyl pendent polysiloxane can be present in a range of from about 10 wt % to about 50 wt % of the curable composition, about 10 wt % to about 30 wt %, or less than, equal to, or greater than about 10 wt %, 11, 12, 13, 14, 15, 16, 17, 18, 19, 20 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 wt %. The vinyl pendent polysiloxane component can include one or more vinyl pendent polysiloxanes. In some embodiments, the polysiloxanes will be identical by way of structure, molecular weight, mole percent of repeating units, or vinyl content. In other embodiments, the vinyl pendent polysiloxane component can include a blend of vinyl pendent polysiloxanes that differ by way of structure, molecular weight, mole percent of repeating units, or vinyl content. In some embodiments the vinyl pendent polysiloxane component comprises one or more vinyl pendent polysiloxane homopolymers, vinyl pendent polysiloxane copolymers, or combinations thereof.

A suitable example of a vinyl pendent polysiloxane is a polysiloxane having a structure according to Formula I:

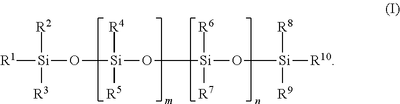

In Formula I, R¹, R², R³, R⁴, R⁵, R⁶, R⁷, R⁸, R⁹, and R¹⁰, are independently chosen from —H, —OH, and substituted or unsubstituted (C₁-C₂₀)hydrocarbyl. At least one of R¹, R⁴, R⁵, and R¹⁰, comprises a vinyl group. Additionally, m and n are in random or block orientation. The units m and n can represent the number of each repeating unit in the polysiloxane. Alternatively, the units m and n can represent the mol % of each repeating unit in the polysiloxane. The unit m can be any positive integer and the unit n can be any positive integer or zero.

In further embodiments, R¹, R², R³, R⁴, R⁵, R⁶, R⁷, R⁸, R⁹, and R¹⁰ of the polysiloxane according to Formula I, can be independently chosen from —H, —OH, substituted or unsubstituted (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxyl, and (C₁-C₂₀)haloalkyl. In further embodiments, the vinyl pendent polysiloxane component can include one or more vinyl pendent polysiloxanes independently having a structure according to Formula II:

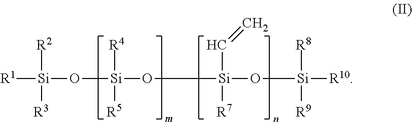

In still further embodiments, the vinyl pendent polysiloxane component includes a vinyl polysiloxane having the structure according to Formula III:

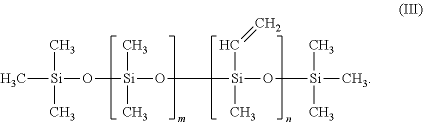

A vinyl content of the one of more vinyl pendent polysiloxanes can be in a range of from about 0.005 mmol/g to about 0.2 mmol/g, about 0.005 mmol/g to about 0.1 mmol/g, or less than, equal to, or greater than about 0.0050, 0.0060, 0.0070, 0.0080, 0.0090, 0.0100, 0.0200, 0.0300, 0.0400, 0.0500, 0.0600, 0.0700, 0.0800, 0.0900, 0.1000, or about 0.2000. The pendent vinyl content of the one or more vinyl polysiloxanes can affect the degree to which the vinyl pendent polysiloxanes cross-link (either with other vinyl pendent polysiloxanes or hydrogen pendent polysiloxanes).

In any formula described herein, the value m can be in a range of from about 300 to about 6,000; about 300 to about 400; about 5,600 to about 5,800; less than, equal to, or greater than about 300; 350; 400; 450; 500; 550; 600; 650; 700; 750; 800; 850; 900; 950; 1,000; 1,100; 1,150; 1,200; 1,250; 1,300; 1,350; 1,400; 1,450; 1,500; 1,550; 1,600; 1,650; 1,700; 1,750; 1,800; 1,850; 1,900; 1,950; 2,000; 2,100; 2,150; 2,200; 2,250; 2,300; 2,350; 2,400; 2,450; 2,500; 2,550; 2,600; 2,650; 2,700; 2,750; 2,800; 2,850; 2,900; 2,950; 3,000; 3,100; 3,150; 3,200; 3,250; 3,300; 3,350; 3,400; 3,450; 3,500; 3,550; 3,600; 3,650; 3,700; 3,750; 3,800; 3,850; 3,900; 3,950; 4,000; 4,100; 4,150; 4,200; 4,250; 4,300; 4,350; 4,400; 4,450; 4,500; 4,550; 4,600; 4,650; 4,700; 4,750; 4,800; 4,850; 4,900; 4,950; 5,000; 5,100; 5,150; 5,200; 5,250; 5,300; 5,350; 5,400; 5,450; 5,500; 5,550; 5,600; 5,650; 5,700; 5,750; 5,800; 5,850; 5,900; or about 5,950.

In any formula described herein, the value n can be in a range of from about 2 to about 50, about 2 to about 40, or less than, equal to, or greater than about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50.

A viscosity of the one or more vinyl pendent polysiloxanes can independently be in a range of from about 700 centistokes (cSt) to about 4,000,000 cSt 25° C., about 10,000 cSt about 300,000 cSt, or less than, equal to, or greater than about 700 cSt; 1,000; 10,000; 100,000; 500,000; 1,000,000; 1,500,000; 2,000,000; 2,500,000; 3,000,000; 3,500,000; or about 4,000,000 cSt 25° C. The viscosity of the vinyl pendent polysiloxane can affect the ability of the vinyl pendent polysiloxane and the hydrogen pendent polysiloxane to mix with each other and form a linked network therebetween. Furthermore, the viscosity of the hydrogen pendent polysiloxane can be chosen to be low enough to facilitate easy application of the composition to the user's skin and increase the perceived comfort of the composition.

The hydrogen pendent polysiloxane component can be in a range of from about 0.5 wt % to about 20 wt % of the composition, about 1.4 wt % to about 3 wt %, or less than, equal to, or greater than about 0.5 wt %, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or about 20 wt %.

The hydrogen pendent polysiloxane component can include one or more hydrogen pendent polysiloxanes. In some embodiments, the polysiloxanes will be identical by way of structure, molecular weight, mole percent of repeating units, or hydrogen content. In other embodiments, the hydrogen pendent polysiloxane component can include a blend of hydrogen polysiloxanes that differ by way of structure, molecular weight, mole percent of repeating units, or hydrogen content. In some embodiments the hydrogen pendent polysiloxane component comprises one or more hydrogen pendent polysiloxane homopolymers, hydrogen pendent polysiloxane copolymers, or combinations thereof. The hydrogen pendent polysiloxane forms part of a cross-linked network in a cured product and can also react with any —OH groups to form hydrogen gas which can foam the cured product.

A suitable example of a hydrogen pendent polysiloxane is a polysiloxane having a structure according to Formula IV:

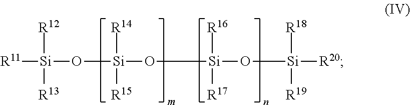

In Formula IV, R¹¹, R¹², R¹³, R¹⁴, R¹⁵, R¹⁶, R¹⁷, R¹⁸, R¹⁹, and R²⁰ are independently chosen from —H, —OH, substituted or unsubstituted (C₁-C₂₀)hydrocarbyl and at least one of R¹¹, R¹⁴, R¹⁵, and R²⁰, is —H. Additionally, p and q are in random or block orientation. The units p and q can represent the number of each repeating unit in the polysiloxane. Alternatively, the units p and q can represent the mol % of each repeating unit in the polysiloxane. The unit p can be any positive integer and the unit q can be any positive integer or zero.

In further embodiments, R¹¹, R¹², R¹³, R¹⁴, R¹⁵, R¹⁶, R¹⁷, R¹⁸, R¹⁹, and R²⁰ of the polysiloxane according to Formula IV are independently chosen from —H, —OH, substituted or unsubstituted (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxyl, and (C₁-C₂₀)haloalkyl. In further embodiments, the hydrogen pendent polysiloxane component can include one or more hydrogen pendent polysiloxanes independently having a structure according to Formula V:

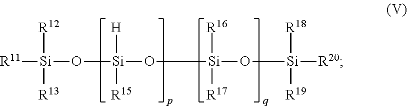

In still further embodiments, the hydrogen pendent polysiloxane component includes a hydrogen pendent polysiloxane having a structure according to Formula VI:

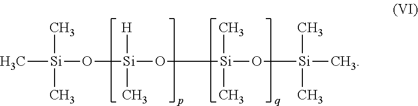

A hydrogen content of the one of more hydrogen pendent polysiloxanes can be in a range of from about 0.0010 mmol/g to about 5 mmol/g, about 0.005 mmol/g to about 0.1 mmol/g, or less than, equal to, or greater than about 0.0010 mmol/g, 0.0020, 0.0030, 0.0040, 0.0050, 0.0060, 0.0070, 0.0080, 0.0090, 0.0100, 0.0200, 0.0300, 0.0400, 0.0500, 0.0600, 0.0700, 0.0800, 0.0900, 0.1000, 0.2000, 0.3000, 0.4000, 0.5000, 0.6000, 0.7000, 0.8000, 0.9000, 1, 1.5, 2, 2.5, 3, 3.5, 4, 4.5, or about 5 mmol/g.

In any of the foregoing formulas, a viscosity of the hydrogen pendant siloxane can be in a range of from about 40 cSt about 50 cSt a temperature of about 25° C., about 42 cSt about 48 cSt, or less than, equal to, or greater than about 40 cSt, 40.5, 41, 41.5, 42, 42.5, 43, 43.5, 44, 44.5, 45, 45.5, 46, 46.5, 47, 47.5, 48, 48.5, 49, 49.5, or about 50 cSt a temperature of about 25° C.

In any of the foregoing formulas, the value p can be between about 3 and about 20, about 5 and about 10, less than, equal to, or greater than about 3, 4, 5, 6, 7, 8, 9, 10, 11, 12, 13, 14, 15, 16, 17, 18, 19, or about 20. The value q can be between about 15 and about 40, about 20 and about 30, less than, equal to, or greater than about 15, 16, 17, 18, 19, 20, 21, 22, 23, 24, 25, 26, 27, 28, 29, 30, 31, 32, 33, 34, 35, 36, 37, 38, 39, or about 40.

In the composition a ratio between the vinyl pendant siloxane and the hydrogen pendant siloxane can be in a range of from about 1:5 to about 1:9, about 1:5 to about 1:7, less than, equal to, or greater than about 1:5, 1:6, 1:7, 1:8, or 1:9. In some embodiments, it was surprisingly found that a ratio of 1:7 yielded a composition that provided exceptional results in reducing the appearance of wrinkles.

The polymerization catalyst can function to catalyze the formation of a crosslinked network of during curing. Examples of suitable polymerization catalysts include platinum containing catalysts that allow the composition to be linked at room temperature, although the rate of linking can be increased by increasing the temperature. The catalyst can be in a range of from about 0.2 wt % to about 4 wt % of the composition, about 0.5 wt % to about 3.3 wt %, less than, equal to, or greater than about 0.2 wt %, 0.3. 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or about 4 wt %. In examples where the polymerization catalyst includes a platinum complex, the polymerization catalyst can be in a range of from about 1 ppm to about 1000 ppm of the curable composition based on the platinum amount, about 10 ppm to about 250 ppm, or less than, equal to, or greater than about 1 ppm, 10, 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 ppm. Examples of suitable platinum containing catalysts include bis(acetylacetonato)platinum(ii), cis-diamminedichloroplatinum(ii), di-μ-chloro-bis[chloro(cyclohexene)platinum(ii)], cis-dichlorobis(triphenylphosphane)platinum(ii), dichloro(cycloocta-1.5-diene)platinum(ii), dihydrogen hexachloroplatinate(iv) hydrate, dihydrogen hexachloroplatinate(iv), platinum(0) divinyltetramethylsiloxane complex, tetrakis(triphenylphosphane)platinum(0), dihydrogen hexachloroplatinate(iv) solution, or mixtures thereof.

The solvent can be chosen from any suitable individual solvent or solvent system. The amount of solvent in the composition can vary, but typically the solvent makes up the balance of the composition to 100 wt %. The solvent can include propylene glycol, isododecane, ethanol, butylene glycol, glycerine, or mixtures thereof. In some embodiments, isododecane and ethanol can be used as the only solvent. In some embodiments, any one of water, propylene glycol, butylene glycol, and glycerine are used together with isododecane.

The composition can include any number of suitable additional components. For example, the composition can further include a pigment, a rheology modifier, a reinforcer, an adhesion promoter, an antifreeze agent, a humectant, an emulsifier, and mixtures thereof. The additional components can individually account for any weight percent of the composition. For example, the individual additional components can range from about 0.5 wt % to about 50 wt % of the composition, about 2 wt % to about 20 wt %, less than, equal to, or greater than about 0.5 wt %, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or about 50 wt %.

The pigment can be any suitable pigment to impart a desired color in the composition. In some embodiments, the composition can be used as a foundation and the color can substantially match the color of a user's skin. Examples of suitable pigment materials include titanium oxide, black iron oxide, red iron oxide, yellow iron oxide, micro talc, ultramarine blue.

The rheology modifier can be used to affect any rheological property of the composition such as its elasticity or viscosity. Examples of suitable rheological modifiers include polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, polyurethanes, polycarboxylic acids, polyvinyl acetates, polyesters, polyamides, polyamines, polyethyleneimines, maleic/(acrylate or methacrylate) copolymers, copolymers of methylvinyl ether and of maleic anhydride, copolymers of vinyl acetate and crotonic acid, copolymers of vinylpyrrolidone and of vinyl acetate, copolymers of vinylpyrrolidone and of caprolactam, vinyl pyrollidone/vinyl acetate copolymers, copolymers of anionic, cationic and amphoteric monomers, karaya gum, tragacanth gum, gum Arabic, acemannan, konjac mannan, acacia gum, gum ghatti, whey protein isolate, and soy protein isolate; seed extracts including guar gum, locust bean gum, quince seed, and psyllium seed; seaweed extracts such as Carrageenan, alginates, and agar; fruit extracts (pectins); those of microbial origin including xanthan gum, gellan gum, pullulan, hyaluronic acid, chondroitin sulfate, and dextran; and those of animal origin including casein, gelatin, keratin, keratin hydrolysates, sulfonic keratins, albumin, collagen, glutelin, glucagons, gluten, zein, shellac, cellulose derivatives such as hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose and other cellulose ethers/esters; guar derivatives such as hydroxypropyl guar; and combinations thereof.

The adhesion promoter can be used to increase the adhesion between the composition and a user's skin or hair. The adhesion promoter can be chosen from any suitable component such as a thermoplastic resin, a glycerol ester of rosin, a resin derived from the esterification of a gum rosin and glycerol, a resin derived from the esterification of a gum rosin and pentaerythritol, a methyl ester of a hydrogenated gum rosin, a blend of sucrose acetate isobutyrate with denatured ethanol, and mixtures thereof.

The humectant can be used to prevent loss of moisture thereby retaining a hair's or skin's natural moisture. Some humectants also have the ability to actively attract moisture. In hair products, the humectant can be used to volumize the hair by attracting moisture which expands the hair shaft. There many suitable humectants that can be used, examples include a protein, an acid, a polysaccharides, a glycerine, a sorbitol, a urea, an extract of aloe vera, an extract of alge, or a mixture thereof. The humectant can also be a propylene glycol or a butylene glycol (which can also function as an antifreeze agent). The emulsifier can be used to mix water and oils to form a homogenous mixture keeping the water and oil in solution. An example of a suitable emulsifier is Tween 20, also known as Polysorbate 20. Tween 20 is a polysorbate surfactant whose stability and relative non-toxicity allows it to be used as a detergent and emulsifier. Tween 20 has a chemical formula of C₅₈H₁₁₄O₂₆, a molar mass of 1227.54 g/mol and a density of 1.10 g/cm³. Another suitable emulsifier includes ABIL EM 90, which is a mixture of cetyl PEG, PPG-10/1 dimethicone. Other emulsifiers include Pemulen E2-4U polymeric emulsifier(a mixture of acrylates/C₁₀-30 alkyl acrylate crosspolymer); Arlcel 165 (a mixture of glyceryl stearate and PEG-100 stearate); TEGO SMOV (a sorbitan oleate); Promulgen G (a mixture of stearyl alcohol and ceteareth-20); Lecigel (a mixture of sodium acrylates copolymer and lecithin)

The antifreeze agent can include a butylene glycerol, ethyl alcohol, ethyl glycol, propylene glycol, glycerin, triglycerin, hexaglycerin, PPG-4, PPG-6, PEG-5, PEG-6, PEG-8, PEG-12, PEG-14, PEG-18, PEG20, Tergitol™ 15-S-9 available from DOW Chemical Company, Midland Mich., and trideceth-12.

The linkable composition can be present as a single phase composition or a multiple phase composition. For example, the linkable composition can include a first phase and a second phase. One reason to have the composition include two phases is that it can allow the user to have better control of the initiation of the curing to form the linked composition. This can be because neither phase is capable of being cured or linked until the phases are combined.

In embodiments where the linkable composition includes a first phase and a second phase, the first phase can include the vinyl pendant siloxane component and the hydrogen pendant siloxane component, the rheology modifier, the reinforcer, the adhesion promoter, and mixtures thereof. The second phase can include the antifreeze agent, the humectant, the catalyst, the solvent, the emulsifier, the pigment, and mixtures thereof. The first phase, the second phase, or both can be in the form of a lotion, cream, paste, or spray. Each phase can be stored in an independent container or dispensing apparatus.

In use, the linkable composition can be applied to a keratin containing substrate. As is understood, keratin is a fibrous protein forming the main structural constituent of skin or hair. Therefore, the keratin containing substrate can be a hair or skin.

The first phase can be contacted with the keratin containing substrate by rubbing or spraying a quantity of the first phase on the substrate. The location to which the first phase is applied can be a location that has a degree of wrinkling thereon. After the first phase is applied to a suitable degree, the second phase is applied right on top of the first phase. It is necessary for the second phase to completely cover the first phase.

Once the first phase is brought into contact with the second phase, the vinyl pendent siloxane component and the hydrogen pendent siloxane component begin to link. The linking forms a linked or cured network which contracts the substrate. In the case of skin, the contraction reduces the appearance of wrinkles by tightening the substrate. According to some embodiments, the visible appearance of wrinkles on skin can be noticeably reduced in as few as four hours. A volume:volume ratio of the first phase and the second phase can be any suitable ratio. In some embodiments, the volume:volume ratio can be in a range of from about 1:1 to about 1:10, about 1:4 to about 1:7, less than, equal to, or greater than about 1:1, 1:2, 1:3, 1:4, 1:5, 1:6, 1:7, 1:8, 1:9, or about 1:10.

The tightening effect of the linked composition can be quantified according to the following method. A piece of flat transparent polyester film having known dimensions can be contacted with second polyester film coated with the cosmetic composition having identical dimensions to the flat transparent polyester film. As the composition links, the second film begins to contract after 10 minutes and is removed after 30 minutes, the second film is removed and measured. A ratio between the second film prior to contraction and after contraction is determined. According to various embodiments, the compositions described herein are capable of achieving a contraction of 10:1 to about 1.15:1, about 5:1 to about 3:1, less than, equal to, or greater than about 10:1, 9:1, 8:1, 7:1, 6:1, 5:1, 4:1, 3:1, 2:1, or about 1.5:1.

### Additional Embodiments

The following exemplary embodiments are provided, the numbering of which is not to be construed as designating levels of importance:

Embodiment 1 provides a linkable cosmetic composition comprising:

a vinyl pendant siloxane component;

a hydrogen pendant siloxane component;

a catalyst; and

a solvent.

Embodiment 2 provides the linkable cosmetic composition of Embodiment 1, wherein the vinyl pendant siloxane component is in a range of from about 10 wt % to about 50 wt % of the composition.

Embodiment 3 provides the linkable cosmetic composition of any one of Embodiments 1 or 2, wherein the vinyl pendant siloxane component is in a range of from about 10 wt % to about 30 wt % of the composition.

Embodiment 4 provides the linkable cosmetic composition of any one of Embodiments 1-3, wherein the hydrogen pendant siloxane component is in a range of from about 0.5 wt % to about 20 wt % of the composition.

Embodiment 5 provides the linkable cosmetic composition of any one of Embodiments 1-4, wherein the hydrogen pendant siloxane component is in a range of from about 1.4 wt % to about 3 wt % of the composition.

Embodiment 6 provides the linkable cosmetic composition of any one of Embodiments 1-5, wherein a ratio of the vinyl pendant siloxane to the hydrogen pendant siloxane in the composition is in a range of from about 1:5 to about 1:9.

Embodiment 7 provides the linkable cosmetic composition of any one of Embodiments 1-6, wherein a ratio of the vinyl pendant siloxane to the hydrogen pendant siloxane in the composition is in a range of from about 1:5 to about 1:7.

Embodiment 8 provides the linkable cosmetic composition of any one of Embodiments 1-7, wherein the vinyl pendant siloxane component comprises a vinyl siloxane having a structure according to Formula I:

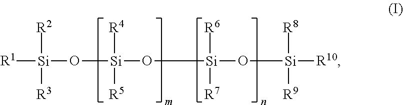

wherein
R¹, R², R³, R⁴, R⁵, R⁶, R⁷, R⁸, R⁹, and R¹⁰, are independently chosen from —H, —OH, and substituted or unsubstituted (C₁-C₂₀)hydrocarbyl;
at least one of R⁴, R⁵, R⁶, and R⁷, comprises a vinyl group;
m is any positive integer;
n is zero or any positive integer; and
m and n are in random or block orientation.

Embodiment 9 provides the linkable cosmetic composition of Embodiment 8, wherein R¹, R², R³, R⁴, R⁵, R⁶, R⁷, R⁸, R⁹, and R¹⁰ are independently chosen from —H, —OH, substituted or unsubstituted (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxyl, (C₁-C₂₀)haloalkyl, and (C₁-C₂₀)haloalkyl.

Embodiment 10 provides the linkable cosmetic composition of any one of Embodiments 8 or 9, wherein the vinyl pendant siloxane component comprises one or more vinyl siloxanes independently having a structure according to Formula II:

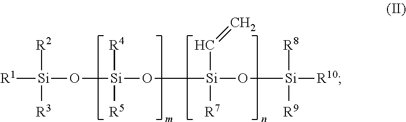

Embodiment 11 provides the linkable cosmetic composition of any one of Embodiments 8-10, wherein the vinyl pendant siloxane component comprises one or more vinyl siloxanes independently having a structure according to Formula III:

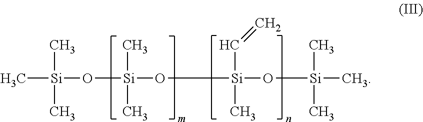

Embodiment 12 provides the linkable cosmetic composition of any one of Embodiments 8-11, wherein the vinyl pendant siloxane component has a vinyl content in a range of from about 0.005 mmol/g to about 0.2 mmol/g.

Embodiment 13 provides the linkable cosmetic composition of any one of Embodiments 8-12, wherein the vinyl pedant siloxane component comprises one or more vinyl siloxane homopolymers, vinyl siloxane copolymers, or combinations thereof.

Embodiment 14 provides the linkable cosmetic composition of any one of Embodiments 8-13, wherein a viscosity of the vinyl pendant siloxane component is in a range of from about 700 cSt about 4,000,000 cSt a temperature of about 25° C.

Embodiment 15 provides the linkable cosmetic composition of any one of Embodiments 8-14, wherein m is between about 300 and about 6000.

Embodiment 16 provides the linkable cosmetic composition of any one of Embodiments 8-15, wherein m is between about 300 and about 400.

Embodiment 17 provides the linkable cosmetic composition of any one of Embodiments 8-16, wherein m is between about 5600 and about 5800.

Embodiment 18 provides the linkable cosmetic composition of any one of Embodiments 8-17, wherein n is between about 2 and about 50.

Embodiment 19 provides the linkable cosmetic composition of any one of Embodiments 8-18, wherein n is between about 2 and about 40.

Embodiment 20 provides the linkable cosmetic composition of any one of Embodiments 1-19, wherein the hydrogen pendant siloxane component comprises a hydrogen siloxane polymer having a structure according to Formula IV:

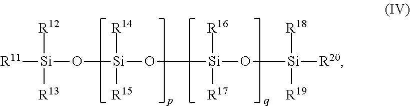

wherein R¹¹, R¹², R¹³, R¹⁴, R¹⁵, R¹⁶, R¹⁷, R¹⁸, R¹⁹, and R²⁰ are independently chosen from —H, —OH, substituted or unsubstituted (C₁-C₂₀)hydrocarbyl and at least one of R¹⁴, R¹⁵, R¹⁶, and R¹⁷, is —H;

p is any positive integer;

q is zero or any positive integer; and

p and q are in random or block orientation.

Embodiment 21 provides the linkable cosmetic composition of Embodiment 20, wherein R¹¹, R¹², R¹³, R¹⁴, R¹⁵, R¹⁶, R¹⁷, R¹⁸, R¹⁹, and R²⁰ are independently chosen from —H, —OH, substituted or unsubstituted (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxyl, (C₁-C₂₀)haloalkyl.

Embodiment 22 provides the linkable cosmetic composition of any one of Embodiments 20 or 21, wherein the hydrogen pendant siloxane component comprises one or more hydrogen siloxane polymers independently having a structure according to Formula V:

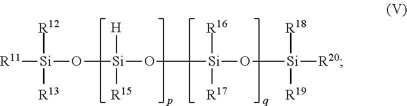

Embodiment 23 provides the linkable cosmetic composition of any one of Embodiments 20-22, wherein the hydrogen pendant siloxane component comprises one or more hydrogen siloxane polymers having a structure according to Formula VI:

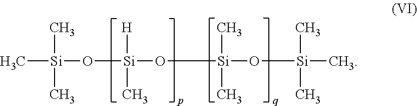

Embodiment 24 provides the linkable cosmetic composition of any one of Embodiments 20-23, wherein the hydrogen pendant siloxane component has a hydrogen content in a range of from about 1.95 mmol/g to about 4.35 mmol/g.

Embodiment 25 provides the linkable cosmetic composition of any one of Embodiments 20-24, wherein the hydrogen pedant siloxane component comprises one or more hydrogen siloxane homopolymers, hydrogen siloxane copolymers, or combinations thereof.

Embodiment 26 provides the linkable cosmetic composition of any one of Embodiments 20-25, wherein a viscosity of the hydrogen pendant siloxane component is in a range of from about 40 cSt about 50 cSt a temperature of about 25° C.

Embodiment 27 provides the linkable cosmetic composition of any one of Embodiments 20-26, wherein p is between about 3 and about 20.

Embodiment 28 provides the linkable cosmetic composition of any one of Embodiments 20-27, wherein p is between about 5 and about 10.

Embodiment 29 provides the linkable cosmetic composition of any one of Embodiments 20-28, wherein q is between about 15 and about 40.

Embodiment 30 provides the linkable cosmetic composition of any one of Embodiments 20-29, wherein q is between about 20 and about 30.

Embodiment 31 provides the linkable cosmetic composition of any one of Embodiments 1-30 wherein the catalyst is in a range of from about 0.2 wt % to about 4 wt % of the composition.

Embodiment 32 provides the linkable cosmetic composition of any one of Embodiments 1-31, wherein the catalyst is in a range of from about 0.5 wt % to about 3.3 wt % of the composition.

Embodiment 33 provides the linkable cosmetic composition of any one of Embodiments 1-32, wherein the catalyst comprises platinum.

Embodiment 34 provides the linkable cosmetic composition of any one of Embodiments 1-33, wherein the catalyst is in a range of from about 1 ppm to about 1000 ppm of the curable composition based on platinum metal.

Embodiment 35 provides the linkable cosmetic composition of any one of Embodiments 1-34, wherein the catalyst is in a range of from about 10 ppm to about 250 ppm of the curable composition based on platinum metal.

Embodiment 36 provides the linkable cosmetic composition of any one of Embodiments 1-35, wherein the catalyst comprises bis(acetylacetonato)platinum(ii), cis-diamminedichloroplatinum(ii), di-μ-chloro-bis[chloro(cyclohexene)platinum(ii)], cis-dichlorobis(triphenylphosphane)platinum(ii), dichloro(cycloocta-1.5-diene)platinum(ii), dihydrogen hexachloroplatinate(iv) hydrate, dihydrogen hexachloroplatinate(iv), platinum(0) divinyltetramethylsiloxane complex, tetrakis(triphenylphosphane)platinum(0), dihydrogen hexachloroplatinate(iv) solution, or mixtures thereof.

Embodiment 37 provides the linkable cosmetic composition of any one of Embodiments 1-36, wherein the catalyst is a platinum(0) divinyltetramethylsiloxane complex.

Embodiment 38 provides the linkable cosmetic composition of any one of Embodiments 1-37, wherein the composition further comprises a pigment, a rheology modifier, a reinforcer, an adhesion promoter, water, an antifreeze agent, a humectant, an emulsifier, and mixtures thereof.

Embodiment 39 provides the linkable cosmetic composition of any one of Embodiments 1-38, wherein the vinyl pendant siloxane component comprises at least two different vinyl pendant siloxanes.

Embodiment 40 provides the linkable cosmetic composition of any one of Embodiments 1-39, wherein the hydrogen pendant siloxane component comprises at least two different hydrogen pendant siloxanes.

Embodiment 41 provides the linkable cosmetic composition of any one of Embodiments 1-40, wherein the solvent comprises propylene glycol.

Embodiment 42 provides the linkable cosmetic composition of any one of Embodiments 1-41, wherein the composition comprises a first phase and a second phase.

Embodiment 43 provides the linkable cosmetic composition of Embodiment 42, wherein the first phase comprises the vinyl pendant siloxane component and the hydrogen pendant siloxane component and the second phase comprises the catalyst and the solvent.

Embodiment 44 provides the linkable cosmetic composition of any one of Embodiments 42 or 43, wherein the first phase further comprises the rheology modifier, the reinforcer, the adhesion promoter, and mixtures thereof.

Embodiment 45 provides the linkable cosmetic composition of any one of Embodiments 42-44, wherein the second phase further comprises the antifreeze agent, the humectant, the catalyst, the solvent, the emulsifier, the pigment, and mixtures thereof.

Embodiment 46 provides the linkable cosmetic composition of any one of Embodiments 42-45, wherein the first phase and the second phase are disposed in separate containers.

Embodiment 47 provides the linkable cosmetic composition of any one of Embodiments 1-46, wherein the vinyl pendant siloxane component comprises one or more vinyl pendant siloxanes independently having a structure according to Formula III:

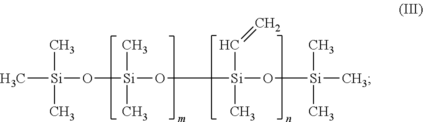

and

the hydrogen pendant siloxane comprises one or more hydrogen pendant siloxane polymers having a structure according to Formula VI:

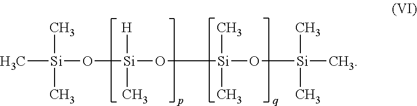

Embodiment 48 provides a substrate comprising keratin having the linkable cosmetic composition disposed thereon.

Embodiment 49 provides a cross-linked composition of the linkable cosmetic composition of any one of Embodiments 1-48.

Embodiment 50 provides a method of using the linkable cosmetic composition of any one of Embodiments 1-49, the method comprising:

disposing a quantity of the composition on a substrate comprising keratin; and

linking the linkable cosmetic composition.

Embodiment 51 provides the method of Embodiment 50, wherein disposing the quantity of the composition comprises:

contacting the first phase of the composition with the substrate comprising keratin; and

contacting the first phase of the composing with the second phase of the composition.

Embodiment 52 provides the method of any one of Embodiments 50 or 51, wherein the substrate comprising keratin is tightened upon linking the linkable cosmetic composition.

Embodiment 53 provides the method of any one of Embodiments 50-52, wherein the method of using comprises a method of reducing the appearance of wrinkles on the substrate comprising keratin.

Embodiment 54 provides the method of any one of Embodiments 50-53, wherein a volume ratio of the first phase and the second phase is in a range of from about 1:1 to about 1:10.

Embodiment 55 provides the method of any one of Embodiments 51-54, wherein the first phase is contacted to the substrate comprising keratin as a paste, a cream, or a lotion.

Embodiment 56 provides the method of any one of Embodiments 51-55, wherein the second phase is contacted to the substrate comprising keratin and the first phase as a paste, liquid or lotion by spraying, dabbing or rubbing.

### EXAMPLES

An anti-wrinkle composition was formed from combining a first phase A and a second phase B. Each of the first phase and the second phase were mixed using a speedmixer to mix the solution at 3,000 rpm for 5 minutes. The constituents of each phase are shown in Table 1.

TABLE 1
Composition of Phase A and Phase B for an antiwrinkle cream

| Phase | INCI Name | Wt % | Function |
|---|---|---|---|
| A | (0.3-0.7% | 20-26 | Film former, |
| | Vinylmethylsiloxane)- | | crosslinking |
| | dimethylsiloxane | | component |
| | copolymer, trimethylsiloxy | | |
| | terminated | | |
| | Methylhydrosiloxane- | 1-4 | Film former, |
| | dimethylsiloxane | | crosslinking |
| | copolymers, trimethylsiloxy | | component |
| | terminated | | |
| | Isododecane | 38-45 | Rheology |
| | | | modifier |
| | Polymethysilsesquioxane | 18-22 | Reinforcer |
| | Hydrophobic fume silica | 5-9 | Reinforcer |
| | Silicone Acrylate | 1-5 | Adhesion |
| | | | promoter |
| B | Deionized water | 20-40 | Water phase |
| | Propylene glycol | 15-25 | Humectant, |
| | | | solvent |
| | Butylene glycol | 5-15 | Humectant, |
| | | | antifreeze |
| | Glycerine | 2-6 | Humectant |
| | Cetyl PEG/PPG-10/1 | 2-6 | Emulsifier |
| | Dimethicone | | |
| | Isododecane | 12-21 | Oil |
| | Nylon-12 | 2-6 | Opacifying |
| | | | Agent |
| | Platinum- | 0.1-1 | Catalyst |
| | divinyltetramethyldisiloxane | | |
| | complex | | |

TABLE 2
Composition of Phase A and Phase B for an antiwrinkle cream

| Phase | INCI Name | wt % | Function |
|---|---|---|---|
| A | (0.3-0.7% | 24.8 | Film former, |
| | Vinylmethylsiloxane)- | | crosslinking |
| | dimethylsiloxane copolymer, | | component |
| | trimethylsiloxy terminated | | |
| | Methylhydrosiloxane- | 1.7 | Film former, |
| | dimethylsiloxane | | crosslinking |
| | copolymers, trimethylsiloxy | | component |
| | terminated | | |
| | Isododecane | 42.2 | Rheology |
| | | | modifier |
| | Polymethysilsesquioxane | 20.9 | Reinforcer |
| | Hydrophobic fume silica | 7 | Reinforcer |
| | Silicone Acrylate | 3.4 | Adhesion |
| | | | promoter |
| B | Deionized water | 34.8 | Water phase |
| | Propylene glycol | 21 | Humectant, |
| | | | solvent |
| | Butylene glycol | 10.1 | Humectant, |
| | | | antifreeze |
| | Glycerine | 4.7 | Humectant |
| | Cetyl PEG/PPG-10/1 | 4.5 | Emulsifier |
| | Dimethicone | | |
| | Isododecane | 19.6 | Oil |
| | Nylon-12 | 4.7 | Opacifying |
| | | | Agent |
| | Platinum- | 0.6 | Catalyst |
| | divinyltetramethyldisiloxane | | |
| | complex | | |

TABLE 3
Composition of Phase A and Phase B for a mascara composition

| Phase | INCI | wt % | Function |
|---|---|---|---|
| A | (0.3-0.7% | 14-18 | Film former, |
| | Vinylmethylsiloxane)- | | crosslinking |
| | Dimethylsiloxane | | component |
| | Copolymer, Trimethylsiloxy | | |
| | Terminated | | |
| | Methylhydrosiloxane- | 1-3 | Film former, |
| | Dimethylsiloxane | | crosslinking |
| | Copolymer, Trimethylsiloxy | | component |
| | Terminated | | |
| | Isododecane | 24-28 | Rheology |
| | | | modifier |
| | Polymethylsilsesquioxane | 12-16 | Reinforcer |
| | Hydrophobic fume silica | 2-6 | Reinforcer |
| | Silicone acrylate | 1-4 | Reinforcer |
| | Cl 77499 | 23-18 | Pigment |
| | Cl 77499 | 4-8 | Pigment |
| B | Water | 32-36 | Water phase |
| | Propylene Glycol | 20-24 | Humectant, |
| | | | solvent |
| | Butylene Glycol | 8-12 | Humectant, |
| | | | antifreeze |
| | Glycerine | 2-6 | Humectant |
| | Cetyl PEG/PPG-10/1 | 2-8 | Emulsifier |
| | Dimethicone | | |
| | Isododecane | 16-22 | Oil |
| | Nylon-12 | 2-6 | Opacifying |
| | | | Agent |
| | Platinum- | 0.2-0.8 | Catalyst |
| | divinyltetramethyldisiloxane | | |
| | complex | | |

TABLE 4
Composition of Phase A and Phase B for a foundation composition

| Phase | INCI | Wt % | Function |
|---|---|---|---|
| A | (0.3-0.7% | 22-26 | Film former, |
| | Vinylmethylsiloxane)- | | crosslinking |
| | Dimethylsiloxane | | component |
| | Copolymer, Trimethylsiloxy | | |
| | Terminated | | |
| | Methylhydrosiloxane- | 1-4 | Film former, |
| | Dimethylsiloxane | | crosslinking |
| | Copolymer, Trimethylsiloxy | | component |
| | Terminated | | |
| | Isododecane | 39-42 | Rheology |
| | | | modifier |
| | Polymethylsilsesquioxane | 18-22 | Reinforcer |
| | Hydrophobic fume silica | 5-9 | Reinforcer |
| | Silicone acrylate | 1-5 | Adhesion |
| | | | promoter |
| B | Titanium Oxide | 2-7 | Pigment |
| | Black Iron Oxide | 0.07-0.10 | Pigment |
| | Red Iron Oxide | 0.15-0.20 | Pigment |
| | Yellow Iron Oxide | 0.40-0.80 | Pigment |
| | Talc | 1.5-3.5 | Pigment |
| | Water | 29-33 | Water phase |
| | Propylene Glycol | 17-22 | Humectant, |
| | | | solvent |
| | Butylene Glycol | 7-11 | Humectant, |
| | | | antifreeze |
| | Glycerine | 2-6 | Humectant |
| | Cetyl PEG/PPG-10/1 | 2-6 | Emulsifier |
| | Dimethicone | | |
| | Isododecane | 16-20 | Rheology |
| | | | modifier |
| | Nylon-12 | 2-6 | Opacifying |
| | | | Agent |
| | Platinum- | 0.30-0.70 | Catalyst |
| | divinyltetramethyldisiloxane | | |
| | complex | | |

1. A linkable cosmetic composition comprising:
a vinyl pendant siloxane component;
a hydrogen pendant siloxane component;
a catalyst; and
a solvent.

2. The linkable cosmetic composition of claim 1, wherein the vinyl pendant siloxane component is in a range of from about 10 wt % to about 50 wt % of the composition.

3. The linkable cosmetic composition of claim 1, wherein the hydrogen pendant siloxane component is in a range of from about 0.5 wt % to about 20 wt % of the composition.

4. The linkable cosmetic composition of claim 1, wherein a ratio of the vinyl pendant siloxane to the hydrogen pendant siloxane in the composition is in a range of from about 1:5 to about 1:9.

5. The linkable cosmetic composition of claim 1, wherein the vinyl pendant siloxane component comprises a vinyl siloxane having a structure according to Formula I: 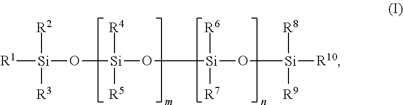
wherein
R¹, R², R³, R⁴, R⁵, R⁶, R⁷, R⁸, R⁹, and R¹⁰, are independently chosen from —H, —OH, and substituted or unsubstituted (C₁-C₂₀)hydrocarbyl;
at least one of R⁴, R⁵, R⁶, and R⁷, comprises a vinyl group;
m is any positive integer;
n is zero or any positive integer; and
m and n are in random or block orientation.

6. The linkable cosmetic composition of claim 5, wherein the vinyl pendant siloxane component comprises one or more vinyl siloxanes independently having a structure according to Formula II:

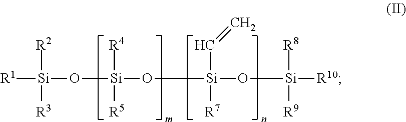

7. The linkable cosmetic composition of claim 1, wherein the hydrogen pendant siloxane component comprises a hydrogen siloxane polymer having a structure according to Formula IV: 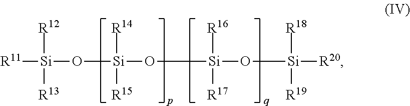
wherein R¹¹, R¹², R¹³, R¹⁴, R¹⁵, R¹⁶, R¹⁷, R¹⁸, R¹⁹, and R²⁰ are independently chosen from —H, —OH, substituted or unsubstituted (C₁-C₂₀)hydrocarbyl and at least one of R¹⁴, R¹⁵, R¹⁶, and R¹⁷, is —H;
p is any positive integer;
q is zero or any positive integer; and
p and q are in random or block orientation.

8. The linkable cosmetic composition of claim 7, wherein the hydrogen pendant siloxane component comprises one or more hydrogen siloxane polymers independently having a structure according to Formula V:

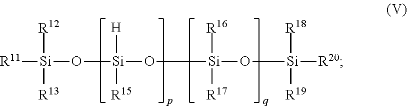

9. The linkable cosmetic composition of claim 1 wherein the catalyst is in a range of from about 0.2 wt % to about 4 wt/o of the composition.

10. The linkable cosmetic composition of claim 1, wherein the catalyst comprises platinum.

11. The linkable cosmetic composition of claim 1, wherein the catalyst is in a range of from about 1 ppm to about 1000 ppm of the curable composition based on platinum metal.

12. The linkable cosmetic composition of claim 1, wherein the composition further comprises a pigment, a rheology modifier, a reinforcer, an adhesion promoter, water, an antifreeze agent, a humectant, an emulsifier, and mixtures thereof.

13. The linkable cosmetic composition of claim 1, wherein the composition comprises a first phase and a second phase.

14. The linkable cosmetic composition of claim 1, wherein
the vinyl pendant siloxane component comprises one or more vinyl pendant siloxanes independently having a structure according to Formula III: 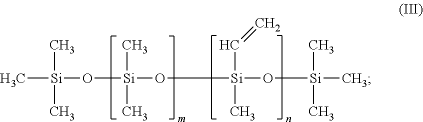
and
the hydrogen pendant siloxane comprises one or more hydrogen pendant siloxane polymers having a structure according to Formula VI:

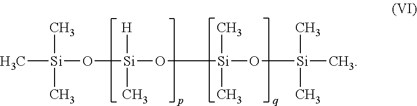

15. A method of using the linkable cosmetic composition of claim 1, the method comprising:
disposing a quantity of the composition on a substrate comprising keratin; and
linking the linkable cosmetic composition.
